(19) United States
(12) Patent Application Publication
(10) Pub. No.: US 2006/0173505 A1
(43) Pub. Date: Aug. 3, 2006

(54) Controlled delivery of electrical pacing therapy for treating mitral regurgitation

(76) Inventors: Rodney W. Salo, Fridley, MN (US); Allan Shuros, St. Paul, MN (US); Joseph M. Pastore, Woodbury, MN (US); Qingsheng Zhu, Wexford, PA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/116,955

(22) Filed: Apr. 28, 2005

Related U.S. Application Data
(63) Continuation-in-part of application No. 11/046,215, filed on Jan. 28, 2005.

(51) Int. Cl. A61N 1/362

(52) U.S. Cl. 607/9

(57) ABSTRACT

A method and apparatus are disclosed for treating mitral or tricuspid regurgitation with electrical stimulation. By providing pacing stimulation to a selected region of the left ventricle, such as one in proximity to the mitral valve apparatus or papillary muscles in a manner that pre-excites the region during early ventricular systole, a beneficial effect is obtained which can prevent or reduce the extent of mitral regurgitation.

### RELATED APPLICATIONS

This application is a continuation-in-part of U.S. patent application Ser. No. 11/046,215, filed on Jan. 28, 2005, the specification of which is hereby incorporated by reference.

### FIELD OF THE INVENTION

This invention pertains to cardiac devices such as pacemakers and other types of devices for treating cardiac dysfunction.

### BACKGROUND

The tricuspid and mitral valves, also referred to as the atrioventricular or AV valves, separate the atrium and ventricle on the right and left sides of heart, respectively. The function of the atrioventricular valves is to allow flow of blood between the atrium and ventricle during ventricular diastole and atrial systole but prevent the backflow of blood during ventricular systole. The mitral valve is composed of a fibrous ring called the mitral annulus located between the left atrium and the left ventricle, the anterior and posterior leaflets, the chordae tendineae, and the papillary muscles. The leaflets extend from the mitral annulus and are tethered by the chordae tendineae to the papillary muscles which are attached to the left ventricle. The function of the papillary muscles is to contract during ventricular systole and limit the travel of the valve leaflets back toward the left atrium. If the valve leaflets are allowed to bulge backward into the atrium during ventricular systole, called prolapse, leakage of blood through the valve can result. The structure and operation of the tricuspid valve is similar.

Mitral regurgitation (MR), also referred to as mitral insufficiency or mitral incompetence, is characterized by an abnormal reversal of blood flow from the left ventricle to the left atrium during ventricular systole. This occurs when the leaflets of the mitral valve fail to close properly as the left ventricle contracts, thus allowing retrograde flow of blood back into the left atrium. Tricuspid regurgitation (TR) occurs in a similar manner. MR and TR can be due to a variety of structural causes such as ruptured chordae tendineae, leaflet perforation, or papillary muscle dysfunction. Functional MR and TR may also occur in heart failure patients due to annular dilatation or myocardial dysfunction, both of which may prevent the valve leaflets from coapting properly.

In acute mitral valve regurgitation, the incompetent mitral valve allows part of the ventricular ejection fraction to reflux into the left atrium. Because the atrium and ventricle are not able to immediately dilate, the volume overload of the atrium and ventricle results in elevated left atrial and pulmonary venous pressures and acute pulmonary edema. The reduction in forward stroke volume due to the reflux through the regurgitant valve reduces systemic perfusion, which if extreme enough can lead to cardiogenic shock. In chronic mitral valve regurgitation, on the other hand, the left atrium and ventricle dilate over time in response to the volume overload which acts as a compensatory mechanism for maintaining adequate stroke volume. The left ventricular dilatation, however, may further prevent proper coaptation of the mitral valve leaflets during systolic ejection, leading to progression of the left ventricular dilatation and further volume overload. Patients with compensated MR may thus remain asymptomatic for years despite the presence of severe volume overload, but most people with MR decompensate over the long term and either die or undergo a corrective surgical procedure. In order to provide early and appropriate intervention, patients with MR may be identified by clinical examination and/or with specific imaging modalities such as echocardiography.

### SUMMARY

A method and apparatus are disclosed for treating mitral or tricuspid regurgitation with electrical stimulation. By providing pacing stimulation to a selected region of the left ventricle, such as one in proximity to the mitral valve apparatus or papillary muscles in a manner that pre-excites the region during early ventricular systole, a beneficial effect is obtained which can prevent or reduce the extent of mitral regurgitation. Such pacing stimulation may be automatically altered by the device in accordance with measurements that are reflective of the severity of the regurgitation.

### BRIEF DESCRIPTION OF THE DRAWINGS

FIGS. 1A and 1B illustrate the mechanisms involved in mitral regurgitation.

FIG. 2 illustrates an exemplary implantable device for delivering pacing therapy to treat mitral or tricuspid regurgitation.

FIG. 3 illustrates the steps involved in employing pacing therapy for treatment of mitral or tricuspid regurgitation in accordance with an exemplary embodiment.

### DETAILED DESCRIPTION

The most common method presently available for definitive treatment of MR is surgical intervention involving repair of the mitral valve or replacement with a mechanical or transplanted valve. In order to provide early and appropriate intervention, patients with MR may be identified by clinical examination and/or with specific imaging modalities such as echocardiography. The present disclosure deals with a method and apparatus for treating mitral (or tricuspid) regurgitation with electrical pacing therapy. Pacing therapy applied in this manner may be used to treat MR either in place of or in addition to conventional surgical or medical options.

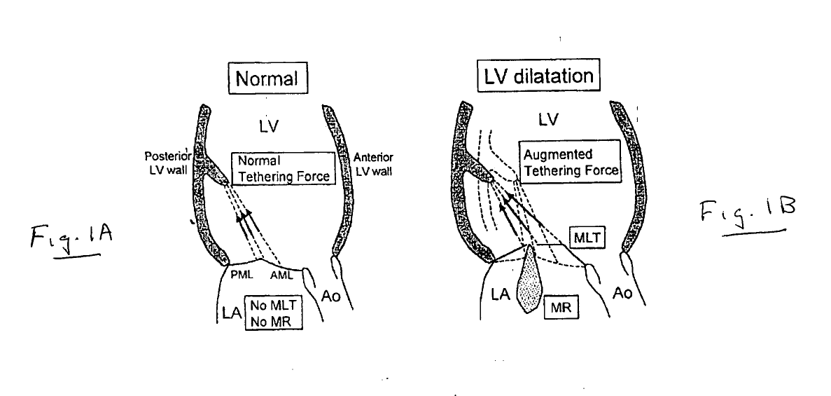

As mentioned above, one mechanism responsible for the development of MR is dilation of the left ventricle, which correspondingly dilates the mitral annulus and/or alters its position, thereby preventing proper coaptation of the valve leaflets. Such ventricular dilation occurs in patients suffering heart failure or subsequent to a myocardial infarction as a compensatory response to decreased cardiac output. Heart failure patients may also suffer from electrical conduction deficits which alter the normal activation patterns of the myocardium during systole. Such electrical conduction deficits may result in abnormal timing of papillary muscle contraction, which also prevents proper leaflet coaptation. FIGS. 1A and 1B are schematic diagrams of the left ventricle LV, left atrium LA, posterior mitral leaflet PML, anterior mitral leaflet AML, aorta AO, papillary muscle PM, and chordae tendineae CT. FIG. 1A illustrates the normal situation during ventricular systole where the posterior and anterior leaflets are tethered by the chordae tendineae and papillary muscle to the posterior wall of the left ventricle in such a manner that the valve leaflets are coapted, thus preventing reflux flow into the atrium. As the ventricle contracts further, corresponding contraction of the papillary muscle maintains the coaptation of the valve leaflets and prevents them from prolapsing into the atrium. FIG. 1B illustrates the situation where the ventricle is abnormally dilated so as to cause mitral regurgitation. The outward displacement of the ventricular walls and papillary muscle causes an augmented tethering force to be applied to the valve leaflets, which prevents proper coaptation and allows reflux flow RF into the atrium. As the ventricle contracts further, simultaneous contraction of the papillary muscle maintains the augmented tethering force and prevents valve closure.

It has been found that pacing therapy may be applied in such a manner that mitral or tricuspid regurgitation is either prevented or lessened in degree in certain patients. In this technique, a pacing electrode is disposed and pacing pulses are delivered so as to pre-excite a specific region of the atrial or ventricular myocardium and result in less regurgitant flow. Early activation of a specific injured region (e.g., an infarcted region) or adjacent areas such as the annulus, papillary muscle, or myocardial region surrounding the valve serves to facilitate coaptation of the valve leaflets, which lessens or prevents regurgitation. This may come about in several different ways. If the ventricular region around the mitral valvular annulus is pre-excited, that ventricular region contracts during the lower afterload pressure that exists during early systole. This may cause the ventricular contraction to constrict the annulus and allow proper coaptation of the valve leaflets to occur. Similarly, pre-excitation of the ventricular region between the valve annulus and the attachment of the papillary muscle to the ventricular wall causes that ventricular region to contract against a lower afterload and lessens the augmented tethering force which prevents proper coaptation of the valve leaflets. Pre-excitation of the papillary muscle can also lessen the augmented tethering force by causing the muscle to be relaxed in later systole and thereby allow valve closure in the dilated ventricle.

In one embodiment, pre-excitation pacing is delivered to a ventricular region in proximity to the mitral or tricuspid valve such that the region is pre-excited during the early phase of ventricular systole. In a patient with intact native atrioventricular conduction, the timing of the pre-excitation may be established with reference to a right or left atrial sense or pace. The atrioventricular delay interval between the atrial sense or pace and the ventricular pre-excitation pace may then be selected to be shorter than the patient's measured intrinsic atrioventricular interval. Because the intrinsic atrioventricular interval varies with heart rate, the intrinsic atrioventricular interval may be measured for a plurality of different heart rate ranges and the atrioventricular delay interval for delivering pre-excitation pacing made to vary accordingly. In a patient either with or without intact native atrioventricular conduction and who is currently receiving conventional bradycardia and/or resynchronization ventricular pacing therapy, the timing of the pre-excitation pacing delivered to a ventricular region in proximity to the mitral or tricuspid valve may be such that the pre-excitation pace occurs before the conventional ventricular pace (or paces), where the latter may be timed with an atrioventricular delay interval selected for optimum hemodynamics. The atrioventricular delay interval for the combination of pre-excitation pacing to the mitral valve region and conventional or resynchronization ventricular pacing may also be made to vary with heart rate.

Described below is an exemplary device that may be used to deliver pre-excitation pacing to the mitral or tricuspid valve region or papillary muscles of the left or right ventricle or to a specific injured region in any of the manners just described. The device is configurable to also deliver conventional bradycardia or resynchronization pacing in addition to the pre-excitation pacing for treating mitral or tricuspid regurgitation. It should be appreciated, however, that a device for delivering pre-excitation pacing to the mitral valve region may possess only those features or components necessary for a particular mode of delivery.

1. Exemplary Device Description

Conventional cardiac pacing with implanted pacemakers involves excitatory electrical stimulation of the heart by the delivery of pacing pulses to an electrode in electrical contact with the myocardium. As the term is used herein, a “pacemaker” should be taken to mean any cardiac device, such as an implantable cardioverter/defibrillator, with the capability of delivering pacing stimulation to the heart, including pre-excitation pacing to the mitral valve region as described herein. A pacemaker is usually implanted subcutaneously on the patient's chest, and is connected to electrodes by leads threaded through the vessels of the upper venous system into the heart. An electrode can be incorporated into a sensing channel that generates an electrogram signal representing cardiac electrical activity at the electrode site and/or incorporated into a pacing channel for delivering pacing pulses to the site.

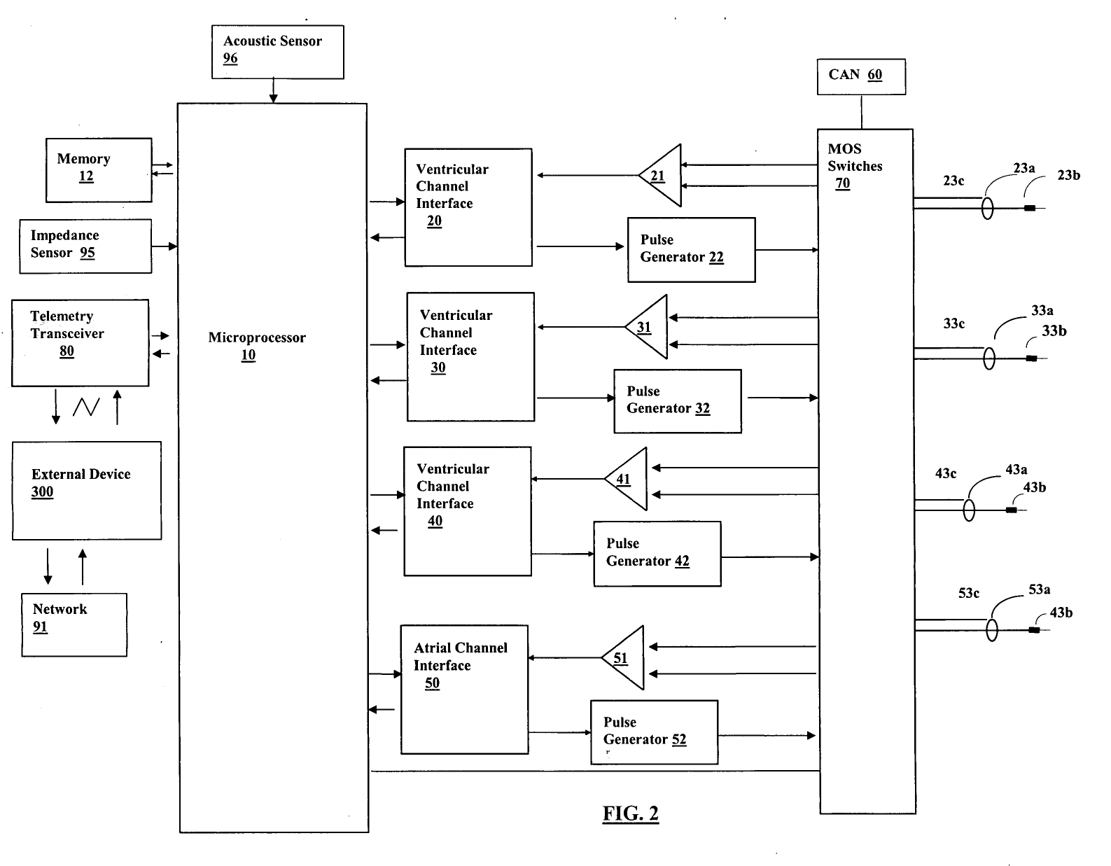

A block diagram of an implantable multi-site pacemaker having multiple sensing and pacing channels is shown in FIG. 2. The controller of the pacemaker is made up of a microprocessor 10 communicating with a memory 12 via a bidirectional data bus, where the memory 12 typically comprises a ROM (read-only memory) for program storage and a RAM (random-access memory) for data storage. The controller could be implemented by other types of logic circuitry (e.g., discrete components or programmable logic arrays) using a state machine type of design, but a microprocessor-based system is preferable. As used herein, the programming of a controller should be taken to refer to either discrete logic circuitry configured to perform particular functions or to the code executed by a microprocessor. The controller is capable of operating the pacemaker in a number of programmed modes where a programmed mode defines how pacing pulses are output in response to sensed events and expiration of time intervals. A telemetry transceiver 80 is provided for communicating with an external device 300 such as an external programmer. An external programmer is a computerized device with an associated display and input means that can interrogate the pacemaker and receive stored data as well as directly adjust the operating parameters of the pacemaker. The telemetry transceiver 80 enables the controller to communicate with an external device 300 via a wireless telemetry link. The external device 300 may be an external programmer that can be used to program the implantable device as well as receive data from it or may be a remote monitoring unit. The external device 300 may also be interfaced to a patient management network 91 enabling the implantable device to transmit data and alarm messages to clinical personnel over the network as well as be programmed remotely. The network connection between the external device 300 and the patient management network 91 may be implemented by, for example, an internet connection, over a phone line, or via a cellular wireless link.

The embodiment shown in FIG. 2 has multiple sensing/pacing channels, where a pacing channel is made up of a pulse generator connected to an electrode while a sensing channel is made up of the sense amplifier connected to an electrode. A MOS switching network 70 controlled by the microprocessor is used to switch the electrodes from the input of a sense amplifier to the output of a pulse generator. The switching network 70 also allows the sensing and pacing channels to be configured by the controller with different combinations of the available electrodes. The channels may be configured as either atrial or ventricular channels allowing the device to deliver conventional ventricular single-site pacing, biventricular pacing, or multi-site pacing of a single chamber, where the ventricular pacing is delivered with or without atrial tracking. In an example configuration, four representative sensing/pacing channels are shown. A right atrial sensing/pacing channel includes ring electrode 53a and tip electrode 53b of bipolar lead 53c, sense amplifier 51, pulse generator 52, and a channel interface 50. A right ventricular sensing/pacing channel includes ring electrode 23a and tip electrode 23b of bipolar lead 23c, sense amplifier 21, pulse generator 22, and a channel interface 20, and a left ventricular sensing/pacing channel includes ring electrode 33a and tip electrode 33b of bipolar lead 33c, sense amplifier 31, pulse generator 32, and a channel interface 30. Another ventricular sensing/pacing channel includes ring electrode 43a and tip electrode 43b of bipolar lead 43c, sense amplifier 41, pulse generator 42, and a channel interface 40. The channel interfaces communicate bi-directionally with a port of microprocessor 10 and include analog-to-digital converters for digitizing sensing signal inputs from the sensing amplifiers, registers that can be written to for adjusting the gain and threshold values of the sensing amplifiers, and registers for controlling the output of pacing pulses and/or changing the pacing pulse amplitude. In this embodiment, the device is equipped with bipolar leads that include two electrodes that are used for outputting a pacing pulse and/or sensing intrinsic activity. Other embodiments may employ unipolar leads with single electrodes for sensing and pacing. The switching network 70 may configure a channel for unipolar sensing or pacing by referencing an electrode of a unipolar or bipolar lead with the device housing or can 60.

The controller controls the overall operation of the device in accordance with programmed instructions stored in memory. The controller interprets electrogram signals from the sensing channels, implements timers for specified intervals, and controls the delivery of paces in accordance with a pacing mode. The sensing circuitry of the pacemaker generates atrial and ventricular electrogram signals from the voltages sensed by the electrodes of a particular channel. An electrogram indicates the time course and amplitude of cardiac depolarization and repolarization that occurs during either an intrinsic or paced beat. When an electrogram signal in an atrial or ventricular sensing channel exceeds a specified threshold, the controller detects an atrial or ventricular sense, respectively, which pacing algorithms may employ to trigger or inhibit pacing. An impedance sensor 95 is also interfaced to the controller for measuring transthoracic impedance. The transthoracic impedance measurement may be used to derive either respiratory minute ventilation for rate-adaptive pacing modes or, as described below, cardiac stroke volume for modulating the delivery of pre-excitation pacing to the mitral valve region.

In order to deliver pre-excitation pacing to a ventricle for treating AV valve regurgitation, one or more pacing channels are configured, each with an electrode disposed near the region to be pre-excited. Sensing channels for the pre-excited region may or may not also be configured. The pre-excitation ventricular pacing may then be delivered in accordance with a conventional atrial tracking bradycardia pacing algorithm (e.g., VDD or DDD) with the atrioventricular delay interval set to a value which results in pre-excitation of the mitral or tricuspid valve region during ventricular systole. As described below, the pacing mode, the pacing configuration, and pacing parameters for optimally treating AV valve regurgitation may be selected in a manner which minimizes regurgitant flow.

Such pre-excitation pacing of the mitral valve region may also be delivered in conjunction with ventricular resynchronization therapy. Ventricular resynchronization therapy is most commonly applied in the treatment of patients with heart failure due to left ventricular dysfunction, which is either caused by or contributed to by left ventricular conduction abnormalities. In such patients, the left ventricle or parts of the left ventricle contract later than normal during systole, which thereby impairs pumping efficiency. In order to resynchronize ventricular contractions in such patients, pacing therapy is applied such that the left ventricle or a portion of the left ventricle is pre-excited relative to when it would become depolarized in an intrinsic contraction. Optimal pre-excitation for treating such a conduction deficit in a given patient may be obtained with biventricular (or multi-site ventricular) pacing or with left ventricular-only pacing. When pre-excitation pacing of the mitral valve region or papillary muscles is delivered in conjunction with ventricular resynchronization therapy, a separate sensing/pacing channel may be used for pre-exciting the mitral valve region. For example, the device illustrated in FIG. 2 could be configured to deliver pre-excitation pacing of the mitral valve location together with biventricular pacing through its three ventricular pacing channels. Because the severity of mitral regurgitation can be affected by increasing heart failure as explained above, it may be beneficial to incorporate ventricular resynchronization pacing into the treatment for mitral regurgitation. For the same reason, treatment of mitral regurgitation may be further enhanced by combining pre-excitation pacing of the mitral valve region with other treatment modalities for heart failure such as left ventricular assist devices, myocardial restraints, and drug therapy.

In one embodiment, the device is programmed to pace the ventricle with the regurgitant valve or papillary muscles at a first programmed AV interval subsequent to an atrial sense or pace and pace the ventricle contralateral to the ventricle with the regurgitant valve at a second programmed AV interval subsequent to an atrial sense or pace. (It should be appreciated that specifying separate AV delay intervals for the two ventricles is equivalent to specifying a biventricular offset interval between right and left ventricular paces.) A patient's intrinsic AV interval between an atrial sense or pace and a sense in the ventricle with the regurgitant valve may be measured, and a programmed AV delay interval which optimally pre-excites the ventricular region in proximity to the regurgitant valve may be computed as a function of the measured intrinsic AV interval.

3. Exemplary Algorithm

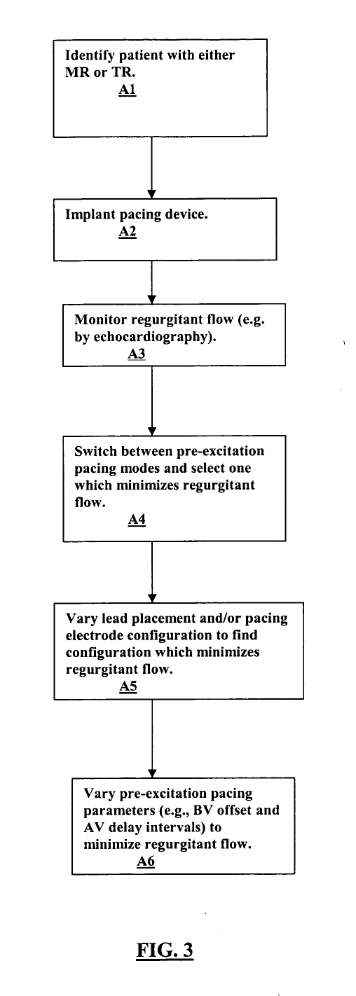

FIG. 3 illustrates an exemplary algorithm for treating AV valve regurgitation with electrical pacing therapy. At step A1, a patient is identified as having either mitral or tricuspid regurgitation by, for example, echocardiography, MRI, or clinical examination. After such identification, the patient is implanted with a pacing device such as that illustrated in FIG. 2 at step A2. After implantation, the patient's regurgitant flow is monitored (e.g., by echocardiography) at step A3. While monitoring the regurgitant flow, one or more adjustments may then be made to the pacing therapy delivered by the device in a manner that minimizes the regurgitant flow. Examples of such adjustments are shown at steps A4 through A6. At step A4, different pacing modes such as atrial pacing, right ventricle-only pacing, left-ventricle-only pacing, biventricular pacing, or other multi-site pacing are tried in order to select the pacing mode which minimizes the regurgitant flow. At step A5, the pacing electrode placement and/or pacing configuration (i.e., which electrodes are used to deliver pacing pulses in a particular mode) are varied in order to the find the electrode placement or configuration that optimally reduces regurgitant flow. Optimal lead placement may be aided by myocardial contrast echocardiography, measuring the electrical impedance between electrodes on a lead in order to measure segmental or global volume changes, or measuring R-wave amplitude with the lead where the bipolar R-wave amplitude decreases as the electrode is advanced toward an infarcted region. At step A6, one or more pre-excitation pacing parameters are varied in order to find those that are most effective in reducing regurgitant flow. Such pre-excitation parameters could include the AV delay interval between an atrial sense or pace and a ventricular pace, the biventricular offset interval between paces delivered to the right and left ventricles, or other inter-pacing site offset interval.

4. Control of Pre-Excitation Pacing

It may be desirable in certain patients to control the delivery of pre-excitation pacing to the mitral valve region or papillary muscles so that such pacing is delivered only when it is needed to lessen mitral regurgitation and/or the amount of pre-excitation delivered is varied in accordance with the severity of the regurgitation. The amount of pre-excitation delivered may be varied by changing the AV delay interval used to pre-excite a myocardial region (e.g., shortening the AV delay to increase the amount of pre-excitation) or by changing the frequency or duration of periodic delivery of pre-excitation pacing.

One way in which the severity of mitral regurgitation may be monitored by an implantable device is via a transthoracic impedance measurement reflective of cardiac stroke volume. As mitral regurgitation produces volume overloading of both the left atrium and ventricle, such monitoring of stroke volume may be used to modulate the frequency or duration of the pre-excitation pacing.

Another way of monitoring the severity of mitral regurgitation is by means of an acoustic sensor 96 incorporated into the implantable device, which allows for changes in heart sounds to be detected. Mitral regurgitation produces predictable changes in the heart sounds produced by valve closure, and these changes become more pronounced as the severity of the regurgitation increases and vice-versa. The S₁ sound, produced by mitral (and tricuspid) valve closure in early systole, diminishes in intensity with mitral regurgitation and becomes less intense as the regurgitation becomes more severe. The S₂ sound, produced by aortic (and pulmonic) valve closure becomes more intense with increasingly severe mitral regurgitation. Because emptying of left ventricle is augmented during systole with mitral regurgitation, the interval between the S₁ and S₂ sounds shortens as mitral regurgitation becomes more severe. Increased intensity of the mitral regurgitation murmur as determined by measuring the loudness of the total acoustic noise in an appropriate frequency range produced during systole may also reflect increased severity of mitral regurgitation. The implantable device may be programmed to determine that changes in heart sounds have occurred by comparing the heart sound measurements with baseline values taken when the extent of the mitral regurgitation is known. When a sufficient change in one or more heart sound intensity or S1-S2 interval measurements occurs, the device may then be programmed to alter the delivery pre-excitation pacing by changing an AV delay value, changing the frequency or duration of periodic pre-excitation pacing, turning the pre-excitation pacing on or off, or switching between a first pacing mode which provides pre-excitation to the mitral valve region and a second pacing mode which does not. The acoustic sensor for monitoring heart sounds may take various forms such as a microphone incorporated into a lead or mounted on the housing of the device, an accelerometer mounted on a lead tip, an accelerometer mounted on or in the device housing, or an accelerometer disposed on an epicardial or endocardial surface.

Another type of acoustic sensor that could be used to monitor the severity of mitral regurgitation is a Doppler ultrasonic transducer mounted on an intravascular lead. The transducer could transmit and receive reflected sound waves that are then processed by the controller in order to monitor the extent of regurgitation through the valve during systole. Regurgitant flow could also be monitored by a pressure transducer positioned in the pulmonary artery or in the left atrium.

It should be appreciated that the techniques and apparatus described above can be used to treat either tricuspid or mitral regurgitation by pre-exciting the regurgitant valve region in either ventricle. If both atrio-ventricular valves are regurgitant, pre-excitation pacing may also be applied to the atrio-ventricular valve region of both ventricles.

Although the invention has been described in conjunction with the foregoing specific embodiments, many alternatives, variations, and modifications will be apparent to those of ordinary skill in the art. Other such alternatives, variations, and modifications are intended to fall within the scope of the following appended claims.

What is claimed is:

1. A method for treating mitral regurgitation, comprising:
delivering pre-excitation pacing to the mitral valve region or papillary muscles;
monitoring one or more heart sounds in order to detect changes reflective of the severity of the mitral regurgitation; and,
altering the delivery of pre-excitation pacing in accordance with detected changes in heart sounds.

2. The method of claim 1 wherein the detected changes in heart sounds include changes in the intensity of S₁.

3. The method of claim 1 wherein the detected changes in heart sounds include changes in the intensity of S₂.

4. The method of claim 1 wherein the detected changes in heart sounds include changes in the S₁-S₂ interval.

5. The method of claim 1 further comprising altering the delivery of pre-excitation pacing in accordance with detected changes in heart sounds by changing an AV delay value.

6. The method of claim 1 further comprising altering the delivery of pre-excitation pacing in accordance with detected changes in heart sounds by changing the frequency of periodic pre-excitation pacing.

7. The method of claim 1 further comprising altering the delivery of pre-excitation pacing in accordance with detected changes in heart sounds by changing the duration of periodic pre-excitation pacing.

8. The method of claim 1 further comprising altering the delivery of pre-excitation pacing in accordance with detected changes in heart sounds by turning the pre-excitation pacing on or off.

9. The method of claim 1 further comprising altering the delivery of pre-excitation pacing in accordance with detected changes in heart sounds by switching between a first pacing mode that provides pre-excitation to the mitral valve region and a second pacing mode which does not.

10. The method of claim 1 further comprising monitoring the extent of regurgitation through the mitral valve during systole with Doppler ultrasound and altering the delivery of pre-excitation pacing in accordance therewith.

11. An implantable cardiac stimulation device, comprising:
an acoustic sensor for monitoring one or more heart sounds in order to detect changes reflective of the severity of the mitral regurgitation;
one or more electrodes adapted to be disposed near a cardiac chamber;
pulse generating circuitry coupled to the one or more electrodes and configured to deliver pacing pulses to a cardiac chamber;
sensing circuitry coupled to the one or more electrodes and configured to detect electrical activity from a cardiac chamber;
a controller coupled to the pulse generating and sensing circuitry and configured to control the delivery of pacing pulses;
an acoustic sensor interfaced to the controller for monitoring one or more heart sounds in order to detect changes reflective of the severity of the mitral regurgitation; and,
wherein the controller is programmed to deliver pacing therapy in a manner which pre-excites a mitral valve region in order to treat mitral regurgitation and further programmed to alter the delivery of pre-excitation pacing in accordance with detected changes in heart sounds.

12. The device of claim 11 wherein the controller is programmed to switch between a first pacing mode which pre-excites a ventricular region in proximity to the regurgitant valve relative to the rest of the ventricle during ventricular systole in order to reduce valve regurgitation and a second pacing mode in accordance with detected changes in heart sounds.

13. The device of claim 11 further comprising:
a Doppler ultrasound transducer disposed on a pacing lead for monitoring regurgitant flow through the mitral valve; and
wherein the controller is programmed to alter the delivery of pre-excitation pacing in accordance with the monitored regurgitant flow.

14. The device of claim 11 further comprising:
a pressure transducer adapted for positioning in the pulmonary artery or in the left atrium for monitoring regurgitant flow through the mitral valve; and
wherein the controller is programmed to alter the delivery of pre-excitation pacing in accordance with the monitored regurgitant flow.

15. The device of claim 11 wherein the detected changes in heart sounds include changes in the intensity of S₁.

16. The device of claim 11 wherein the detected changes in heart sounds include changes in the intensity of S₂.

17. The device of claim 11 wherein the detected changes in heart sounds include changes in the S₁-S₂ interval.

18. The device of claim 11 wherein the controller is programmed to alter the delivery of pre-excitation pacing in accordance with detected changes in heart sounds by changing an AV delay value.

19. The device of claim 11 wherein the acoustic sensor is an accelerometer.

20. The device of claim 11 wherein the acoustic sensor is a microphone.
